(19) United States
(12) Patent Application Publication
(10) Pub. No.: US 2009/0265016 A1
(43) Pub. Date: Oct. 22, 2009

(54) MATERIAL FOR SURGICAL USE IN TRAUMATOLOGY

(76) Inventors: Lasse Daniel Efskind, Nesbru (NO); Tore Etholm Idsoe, Nesoya (NO)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 12/426,611

(22) Filed: Apr. 20, 2009

Related U.S. Application Data
(63) Continuation-in-part of application No. 12/106,605, filed on Apr. 21, 2008.

(51) Int. Cl. A61F 2/28

(52) U.S. Cl. 623/23.51

(57) ABSTRACT

The present invention relates to a material, structure, and method for surgical use in traumatology. More particularly, the present invention relates to a composite material, a temporary biocompatible support structure, and related methods of use of the same in aiding osteosynthesis during healing of a bone fracture. The material keeps its strength in a solid phase in vivo and, to aid removal upon healing, can be transformed into a substantially fluid phase, including, for example, a pulverized state, by the application of energy at a chosen time.

### CROSS-REFERENCE TO RELATED APPLICATIONS

This application is a continuation-in-part application of U.S. Ser. No. 12/106,605, filed Apr. 21, 2008.

### FIELD OF THE INVENTION

The present invention relates to a material, structure, and method for surgical use in traumatology. More particularly, the present invention relates to a composite material, a temporary biocompatible support structure, and related methods of use of the same in supporting the healing process of bone fractures.

### BACKGROUND OF THE INVENTION

Plates, nails, and other structures of various materials are widely used around the world today in bone fracture surgery and healing. Typically, such internal fixation devices are inserted during a first surgery to maintain stability and alignment during the healing process of the fracture. As the fracture heals, a large proportion of these devices must be removed with a second surgery that poses undesirable risks and costs.

Additionally, the rigidity of many internal fixation devices may pose problems during healing. Plates left in position after healing of the fracture can cause local osteoporoses and, hence, weakening of the bone. Plates and screws may also cause soft tissue irritation or progressive foreign body reactions when kept in place for long periods of time after bone healing. Further, as one can experience with bone marrow nailing or cast treatment of fractures, it is not necessary to have absolute rigidity during fracture healing. Depending on whether good alignment and positioning are secured, healing can occur more rapidly with some movements or pulsations in the fracture zone during healing.

A number of prior art devices involve the use of biodegradable, dissolvable, or reabsorbable materials. For example, dissolvable plates using the same materials used in sutures and pins of biodegradable polymers (e.g., polylactide (PLA), polyglycolide (PGA), and other aliphatic polyesters such as polydioxanone (PDS)) are sometimes used. These materials are not entirely satisfactory, however, because they gradually weaken and the rate of absorption or dissolution cannot be actively controlled in vivo. Hence, the time and duration of fixation with bioabsorbable materials cannot be varied or controlled in vivo, which often is necessary as healing time varies widely from patient to patient and is difficult to predict.

For example, U.S. Pat. No. 4,329,743 to Alexander et al. teaches a composite of a bio-absorbable polymer—such as PGA, PLA, and the like—and at least one substrate of a plurality of carbon fibers suitable for constructing a surgical “scaffold” (i.e., a supporting framework) for the growth of new tissue in ligaments, tendons, and bones. A carbon fiber scaffold is enveloped in a bio-absorbable polymer to prevent the migration of the filamentous carbon after implantation. The rate of absorption of the bio-absorbable polymer is meant to coincide with the rate of new tissue growth to enable a transference of load from the carbon fiber-polymer composite to the new tissue over extended periods of time. The material can be used in the construction of bone fixation plates that are applied to bone fractures with bio-compatible screws or other securing means according to standard surgical techniques. However, as explained above, there is a need for a treatment that does not require a second surgery to remove an inserted structure and for a material that will not gradually weaken and that can be more actively controlled in vivo.

Similarly, U.S. Pat. No. 4,496,446 to Ritter et al. teaches structural surgical elements made from bio-absorbable materials having a glycolic ester linkage, such as PGA. The rate of strength loss and degradation in vivo of such polymers is altered by the use of fillers, such as barium sulfate, and by irradiation. In this manner, in vivo control over the rate of disintegration of the polymer is not permitted.

U.S. Pat. No. 5,820,608 to Luzio et al. teaches medical devices, such as stents, catheters, and cannula components, plugs, and constrictors, made of a dissolvable, ionically crosslinked polymer. The devices disintegrate in vivo at a desired time by exposure to a chemical trigger that displaces the crosslinking ion in the crosslinked material through binding or replacement with a non-crosslinking ion. Although the triggered disintegration eliminates the time uncertainty of naturally bioerodible materials from one patient to the next, there is inherent uncertainty in administering the triggering agent, whether by diet, direct local application, parenteral feeding, etc. Also, these materials do not have sufficient strength to be used in fracture healing, and it is generally impractical or impossible to inject a chemical trigger to the entire surface of plates and screws in vivo.

U.S. Pat. No. 5,827,289 to Reiley et al. teaches a balloon for use in compressing cancellous bone and marrow against the inner cortex of bones. When inserted, the inflated balloon forms a cavity in the cancellous bone which can then be filled with antibiotics, bone growth factors, plastic polymers, and other materials, such as those in accordance with the present invention, for treatment of fractures. Reiley teaches a device for use in creating a cavity within bone and contemplates the introduction of flowable materials into the cavity. This is done under high pressure to create a hard and stable format. However, due to the high pressure, this solution is vulnerable to fatal leakages during the up to 18-month-long healing process.

Accordingly, there is a need for a material suitable for surgical use in the internal fixation of bone fractures that can be controlled and reshaped in vivo for fast and easy removal at a chosen time and that does not require surgical intervention for such removal after sufficient healing.

### SUMMARY OF THE INVENTION

The present invention relates to a composite material for surgical use in bone fractures including: a first component comprising a biocompatible polymer matrix capable of being transformed in vivo into a substantially fluid phase (including, for example, a pulverized state) by absorbing energy (as the polymer matrix may or may not itself absorb energy), and a second component capable of strengthening the biocompatible polymer matrix and/or absorbing energy. Embodiments of the invention may include additional components capable of strengthening the biocompatible polymer matrix and/or absorbing energy.

The present invention also relates to a temporary biocompatible support structure for aiding bone fracture osteosynthesis in a living organism comprising: a polymer matrix, and at least one component capable of strengthening said polymer matrix, wherein said support structure is attached to a bone in a living organism, wherein said support structure is substantially solid at a body temperature of said living organism and is substantially fluid when heated to a temperature above said body temperature in vivo, and wherein said support structure is removable at a chosen time in a substantially fluid phase from said living organism.

The present invention relates to a temporary biocompatible support structure for aiding bone fracture osteosynthesis in a living organism comprising: a polymer matrix, and at least one component capable of strengthening said polymer matrix, wherein said support structure is attached to a bone in a living organism, wherein said support structure is substantially solid when applied to said living organism and said support structure can be transformed in vivo into a pulverized state by the absorption of energy, and wherein said support structure is removable in said pulverized state from said living organism.

The present invention further relates to a method for aiding osteosynthesis in bone fracture healing in a living organism comprising the steps of: providing a temporary biocompatible support structure for a bone in a living organism wherein said support structure is substantially solid at a body temperature of said living organism; attaching said support structure to a bone in vivo; applying an energy source to said support structure; and removing a substantial portion of said support structure in a substantially fluid phase from said living organism.

### BRIEF DESCRIPTION OF THE DRAWINGS

FIG. 1 is a cross-sectional anterior view of a temporary biocompatible composite support structure applied to treat a fracture of the right proximal femur of a human in accordance with an embodiment of the present invention.

FIG. 2 is a further view, also cross-sectional, of FIG. 1.

FIG. 3 is a further view, also cross-sectional, of FIGS. 1 and 2.

FIG. 3a is a cross-sectional anterior view of a temporary biocompatible composite support structure applied to treat a fracture of the right proximal femur of a human similar to FIG. 3 and specifically depicting the support structure in a pulverized state, in accordance with an embodiment of the present invention.

FIG. 4 is a further view, also cross-sectional, of FIGS. 1, 2, and 3.

FIG. 5 is a cross-sectional anterior view of a temporary biocompatible composite support structure applied to treat a fracture of the right humerus of a human with a plate fixation in accordance with an embodiment of the present invention.

FIG. 6 is a cross-sectional anterior view of a temporary biocompatible composite support structure applied to treat a fracture of the right proximal femur of a human in accordance with an embodiment of the present invention.

FIG. 7 is a cross-sectional anterior view of a temporary biocompatible composite support structure applied to treat a fracture of the right proximal femur of a human in accordance with an embodiment of the present invention.

FIG. 8 is a further view, also cross-sectional, of FIG. 7.

### DETAILED DESCRIPTION OF THE INVENTION

This description of preferred embodiments is intended to be read in connection with the accompanying drawings, which are to be considered part of the entire written description of the invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. References to axial dimensions and directions (e.g., in an “X” direction, over a “Y” dimension, etc.) should also be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “joined,” “connected,” and “interconnected,” refer to a relationship wherein structures are secured or attached to one another either directly or indirectly through intervening structures, as well as both movable or rigid attachments or relationships, unless expressly described otherwise.

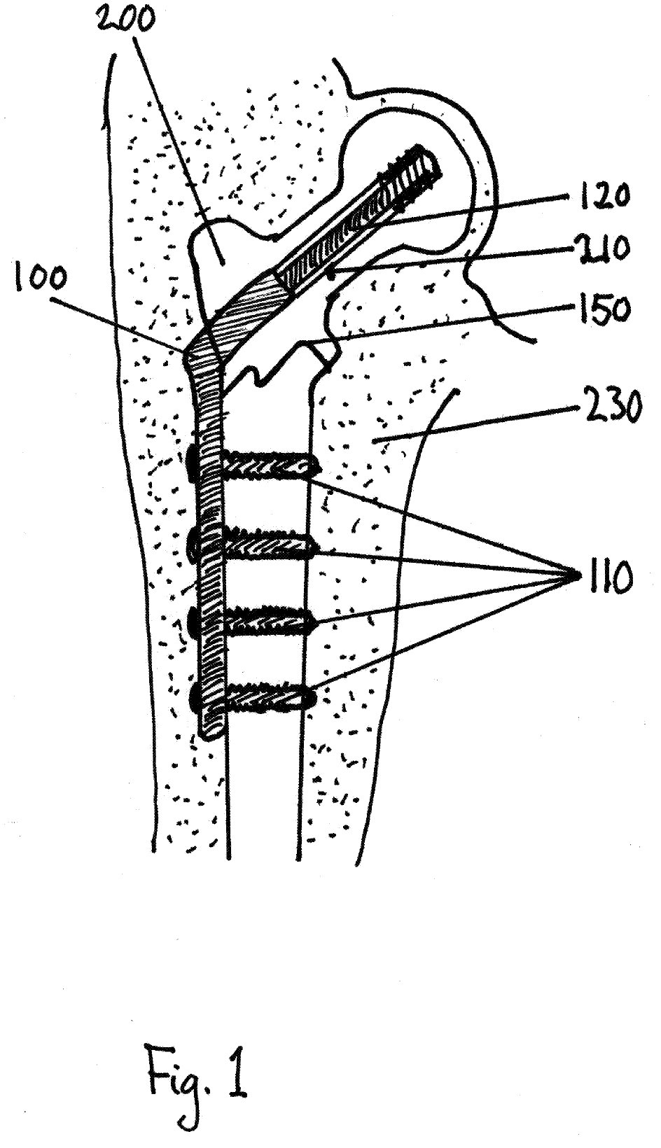

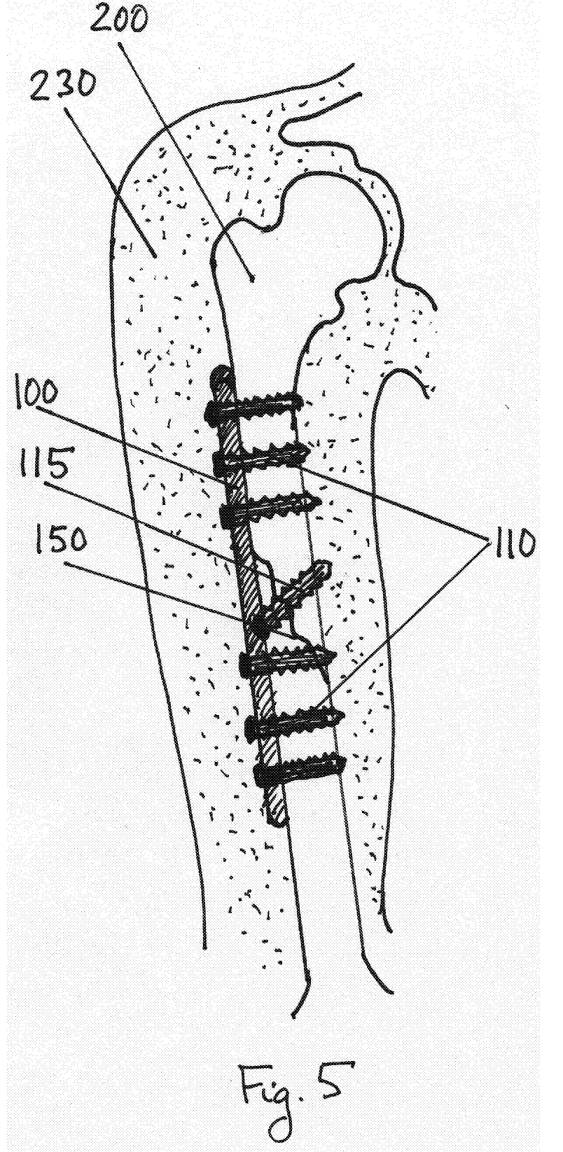

Referring to FIG. 1, an embodiment of the invention includes a temporary biocompatible composite support structure 100 applied to stabilize a fracture 150 of a human bone 200. Soft tissue 230 surrounds bone 200. The support structure 100 is used as an osteosynthesis-aiding device. In some embodiments, support structure 100 is partly inserted into a cavity 210 and partly fixed externally to the bone 200 by fasteners 110. Cavity 210 may be drilled or may otherwise be formed in cancellous bone or marrow. Alternatively, the support structure 100 can be a plate which lies adjacent a fracture 150 of bone 200, as shown in FIG. 5. In other words, the support structure may be disposed on a bone and/or disposed within a bone. In these and other embodiments, support structure 100 is pre-fabricated from a composite material and applied to the fracture area in a substantially solid form in a fashion similar to the traditional use and application of metal or other solid support structures.

In the context of different embodiments, support structure 100 may variously be referred to as an orthopedic device or implant, an internal fixation device, a prosthetic device, and the like.

Support structure 100 is made of at least one composite material for surgical use in traumatology and includes at least two components. The first component includes a polymer matrix. A second component, sometimes referred to as a “reinforcement,” is added to strengthen the polymer matrix and/or to absorb energy such as, for example, heat or shock waves, at a chosen time. A third, fourth or more component capable of strengthening the polymer matrix and/or absorbing energy may also be added. All components are non-toxic and biocompatible. Fasteners 110 and 120 may be made of the same, a similar, or a different material. In some embodiments, support structure 100 includes more than one composite material, each composite material having different desirable properties.

The polymer matrix of the composite material of support structure 100 is a non-toxic, biocompatible polymer matrix suitable for in vivo use in living organisms, such as mammals and, particularly, human beings. At normal ambient pressure, the polymer matrix preferably exists in a substantially solid phase at temperatures approximately within the viable range of normal core body temperatures of the subject organism. For example, a suitable polymer matrix for insertion into a mammal, such as a human, preferably exists in a substantially solid phase at temperatures approximately within the range of about 34° C.-42° C., inclusive. Alternatively, the polymer matrix may alone exist in other than a substantially solid phase at such temperatures where, upon addition of other components or additives, the composite comprising the polymer matrix exists in a substantially solid phase at such temperatures.

At normal ambient pressure, the polymer matrix preferably exists in a substantially fluid phase at temperatures approximately greater than the viable range of normal core body temperatures of the subject organism, but less than a temperature substantially damaging to the cells or body tissue, such as epithelium, connective tissue, muscle tissue, and nervous tissue, of the subject organism. For example, a suitable polymer matrix for insertion into a human preferably exists in a substantially fluid phase at temperatures approximately greater than about 42° C. and less than about 50° C. Alternatively, the polymer matrix may alone exist in other than a substantially fluid phase at such temperatures where, upon addition of other components or additives, the composite comprising the polymer matrix exists in a substantially fluid phase at such temperatures. The polymer matrix, with or without other components or additives, may exist in a substantially fluid phase, or other phases, at temperatures greater than the lower threshold of a temperature substantially damaging to the cells of the subject organism.

As used herein, a “substantially fluid phase” includes, but is not limited to, multiphasic systems exhibiting substantially fluid properties. For example, a biphasic colloid with substantially fluid properties, a multiphasic system comprising several immiscible liquid phases (e.g., an aqueous phase and an organic phase), and a multiphasic system comprising a gaseous phase and a liquid phase are considered to be in a “substantially fluid phase.” Additionally, a pulverized state, e.g., comprising solid granular, flaky, or other particulate matter, is also considered to be a “substantially fluid phase.”

In a preferred embodiment, the polymer matrix has a melting point, melting phase, or melting range at a temperature or over a range of temperatures that is or are greater than about 37° C. and less than a temperature substantially damaging to human body tissue. Most preferably, the polymer matrix has a melting point, melting phase, or melting range at a temperature substantially within a range of about 45° C. to about 50° C., inclusive.

The polymer matrix may include one or more polymers. Suitable polymers include, by way of example and not of limitation: (1) poly(propylene glycol)-block-poly(ethylene glycol)-block-poly(propylene glycol)-bis(2-aminopropyl ether) (PPG-PEG-PPG); (2) poly(ethylene-co-methyl acrylate-co-glycidyl methacrylate) (PEGMAGMA); (3) poly(ethylene adipate), tolylene 2,4-diisocyanate terminated (PEAcy); (4) poly(ethylene glycol) (PEG); (5) poly(ethylene glycol) dimethyl ether (PEGdme); (6) poly(ethylene glycol) distearate (PEGds); (7) poly(propylene carbonate) (PPC); (8) poly(ethylene oxide) (PEO); (9) poly(vinyl acetate) (PVAc).

Other suitable polymers for the polymer matrix include polycaprolactone (PCL) and polycaprolactone polyols.

The second component is added to the first component including the polymer matrix to strengthen the polymer matrix. In some embodiments, the second component includes non-toxic particles, flakes, or fibers, such as, for example, clay, metal flakes, or carbon, mineral, or glass fibers. In other embodiments, the second component includes nanostructures. The nanostructures may be, for example, nano-tubes, nano-rods, or nano-particles. These nanostructures may be made of, for example, carbon, metal, or oxides of metal. In some embodiments, the second component includes nanostructures made of manganese dioxide (MnO₂), titanium dioxide (TiO₂), or low density metals, such as flakes or fibers of magnesium or aluminum.

In some embodiments, the second component is nano-clay.

Preferably, the second component of the composite material is also capable of absorbing energy. The second component may be capable of absorbing energy, such as heat, via, for example, conduction, convection, or electromagnetic radiation, or it may be capable of absorbing shock waves. As the second component absorbs energy, the polymer matrix transforms in vivo into a substantially fluid phase, including, for example, a pulverized state. In some embodiments, the second component absorbs thermal energy or heat and the polymer matrix transforms in vivo into a substantially fluid phase when it reaches a temperature sufficiently greater than the normal core body temperature of the subject organism, as explained above. In preferred embodiments, the polymer matrix enters a substantially fluid phase at a temperature approximately greater than about 45° C. and less than about 50° C. In other embodiments, the second component absorbs shock waves and the polymer matrix transforms in vivo into a pulverized state exhibiting substantially fluid properties. In this manner, the composite material may be quickly degraded in a manner similar to that used in extracorporeal shock wave lithotripsy (ESWL), which is commonly used to break apart kidney stones.

In some embodiments, a third component is added to the first component and the second component of the composite material to further strengthen the polymer matrix and/or to increase its ability to absorb energy. In some embodiments, the third component includes non-toxic particles, flakes, or fibers, such as, for example, clay, metal flakes, or carbon, mineral, or glass fibers. In other embodiments, the third component includes nanostructures. The nanostructures may be, for example, nano-tubes, nano-rods, or nano-particles. These nanostructures may be made of, for example, carbon, metal, or oxides of metal. In some embodiments, the third component includes nanostructures made of manganese dioxide (MnO₂), titanium dioxide (TiO₂), or low density metals, such as flakes or fibers of magnesium or aluminum.

As with the second component, the third component may also be capable of absorbing energy, such as heat, via, for example, conduction, convection, or electromagnetic radiation, or it may be capable of absorbing shock waves. As the third component absorbs energy, the polymer matrix transforms in vivo into a substantially fluid phase, including, for example, a pulverized state.

Other components, such as a fourth component, a fifth component, and so on, may also be added to strengthen the polymer matrix and/or absorb energy, or to impart any other of a number of desirable properties to the support structure. Alternatively, one or more of these components may be added to facilitate dispersion into the polymer matrix.

With the addition to the first component including the polymer matrix of the second component and, in some embodiments, a third or more components, the composite material comprising all three or more components has a strength, tensile strength, stiffness, and/or flexural strength in a substantially solid phase that is comparable to or better than commonly-used osteosynthesis materials, such as other metals or alloys commonly employed in orthopedics, such as, for example, titanium and its alloys, stainless steel, and cobalt-chromium alloys. In other words, the composite material in a substantially solid phase has strength sufficient to support a bone. The composite may be comparable to metal in strength, but may have a physical flexibility closer to the flexibility of bone. This comparable strength or function is desirable so that the composite material can be an adequate substitute for metal as used in temporary fixation devices, such as osteosynthesis materials, and biocompatible support structures made and used in accordance with the present invention. The composite material is also resistant to fatigue, has some elasticity, and is not toxic. An enhanced resistance to fatigue as compared to metal is desirable to improve the suitability of the composite material for osteosynthesis.

In some embodiments, the composite material, as used to form a support structure for bones, is prefabricated in its solid phase, sterilized by irradiation or other low temperature methods, and prepared for use in a living organism. In such embodiments, the support structure is applied to a fracture within the subject organism via a surgical operation. As shown in FIG. 1, an embodiment of the invention includes a temporary biocompatible composite support structure 100 applied to stabilize a fracture 150 of a human bone 200 surrounded by soft tissue 230. Support structure 100 is partly inserted into a cavity 210 and secured by fastener 120 and partly fixed externally to the bone 200 by fasteners 110.

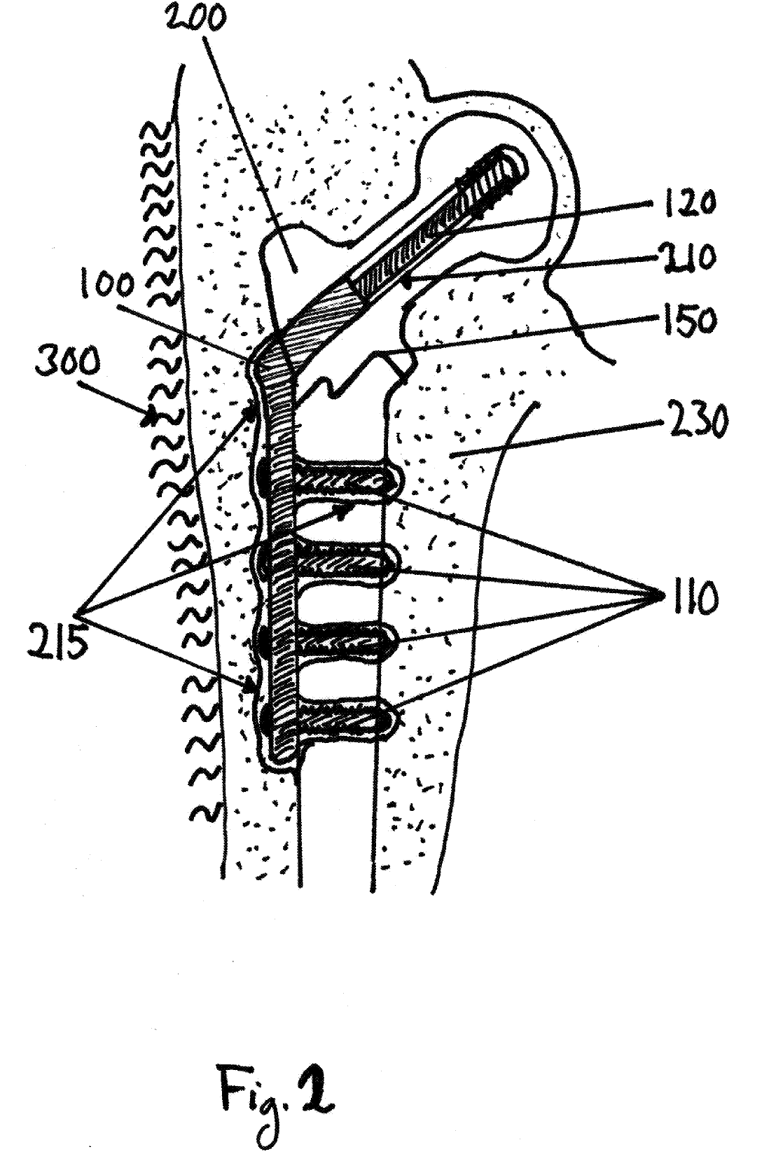

Referring to FIG. 2, after fracture 150 (from FIG. 1) has healed sufficiently to remove support structure 100, the support structure is transformed from a substantially solid phase to a substantially fluid phase (such as a pulverized state) by the application of energy 300 at a chosen time. Sufficient healing of the fracture may occur, for example, in anywhere from 2 months to 18 months after the bone was fractured. Energy 300 is transferred to support structure 100 as, for example, directed electromagnetic radiation of a specific frequency or wavelength to be efficiently absorbed by the composite material of the support structure 100 and with minimal absorption by the surrounding soft tissue 230 or bone 200. In this embodiment, support structure 100 absorbs energy 300 and begins to melt, thereby entering a substantially fluid phase. As support structure 100 enters a substantially fluid phase, its outer surface recedes from the interior surface of cavity 210 within bone 200 and from a pseudo-membrane 215 that forms within soft tissue 230 outside bone 200. Fasteners 110 and 120 can also be converted from a substantially solid phase into a substantially fluid phase, or they can be left in place partially or entirely, for example, if screws made of metal or a different material are employed.

In some embodiments, support structure 100 comprises a composite material including a first component including a polymer matrix and a second component including nanostructures of carbon. As energy 300 is applied, the nanostructures of carbon absorb the energy and the polymer matrix is transformed into a substantially fluid phase. Upon removal of the substantially fluid composite material, some of the carbon fibers may be left in the body.

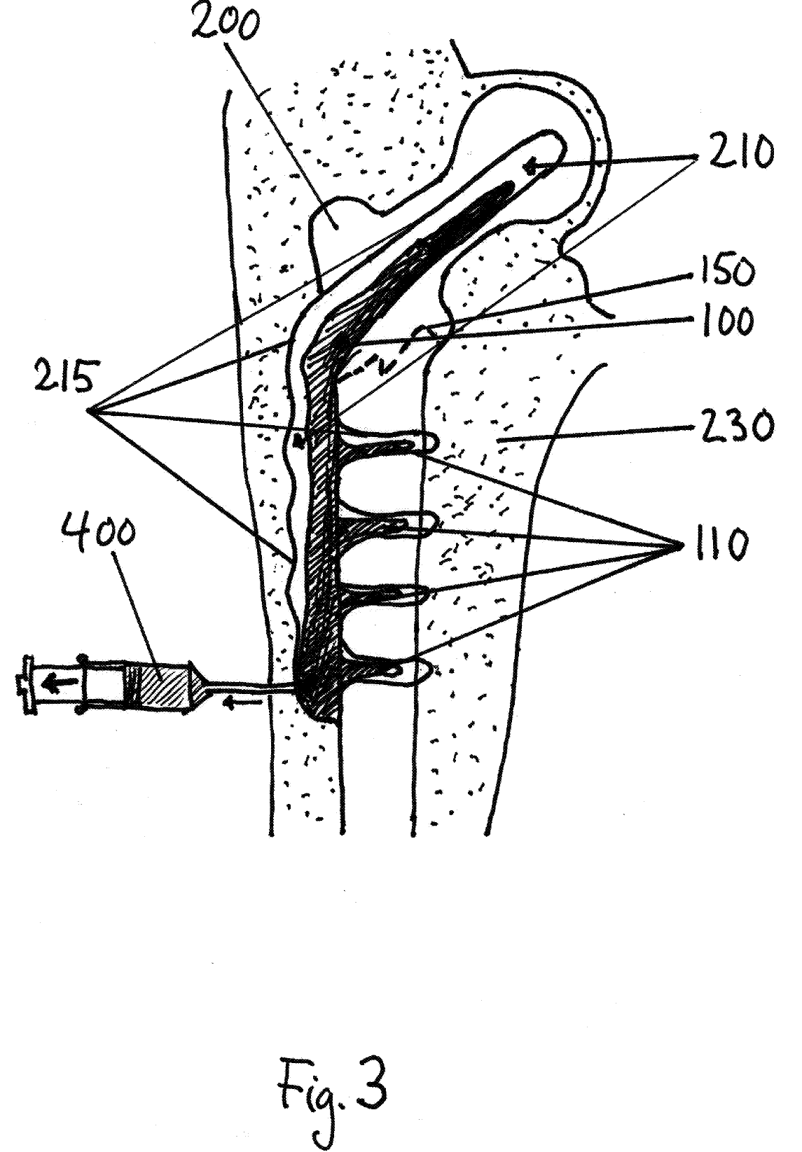

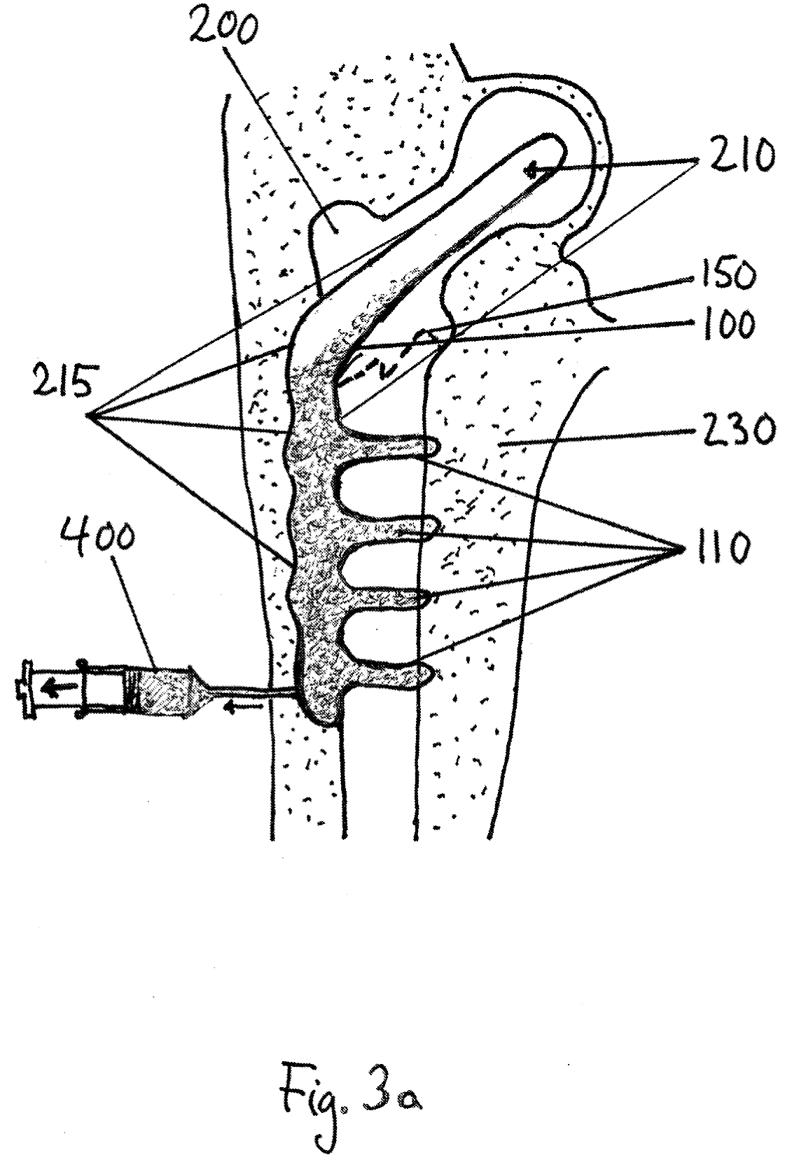

Referring to FIGS. 3 and 3a, the support structure 100, now substantially fluid after absorbing energy 300 (energy 300 is shown in FIG. 2), is removed from cavity 210 in bone 200 and from the space defined by pseudo-membrane 215 in soft tissue 230 via suction and/or flushing applied by a needle or syringe 400. The substantially fluid support structure 100 and fasteners 110 and 120 may be removed by suction and/or flushing through, e.g., a needle, syringe, or special portal. Support structure 100 and fasteners 110 and 120 may be removed in a fast, low-cost, low-risk, and relatively painless procedure that involves minimal or no intervention, such as a surgical intervention. Removal may or may not require the use of anesthesia. Removal of the substantially fluid support structure 100 may be done policlinically, i.e., as an out-patient procedure. FIG. 3a is included to specifically depict an embodiment wherein support structure 100 is transformed into a pulverized state.

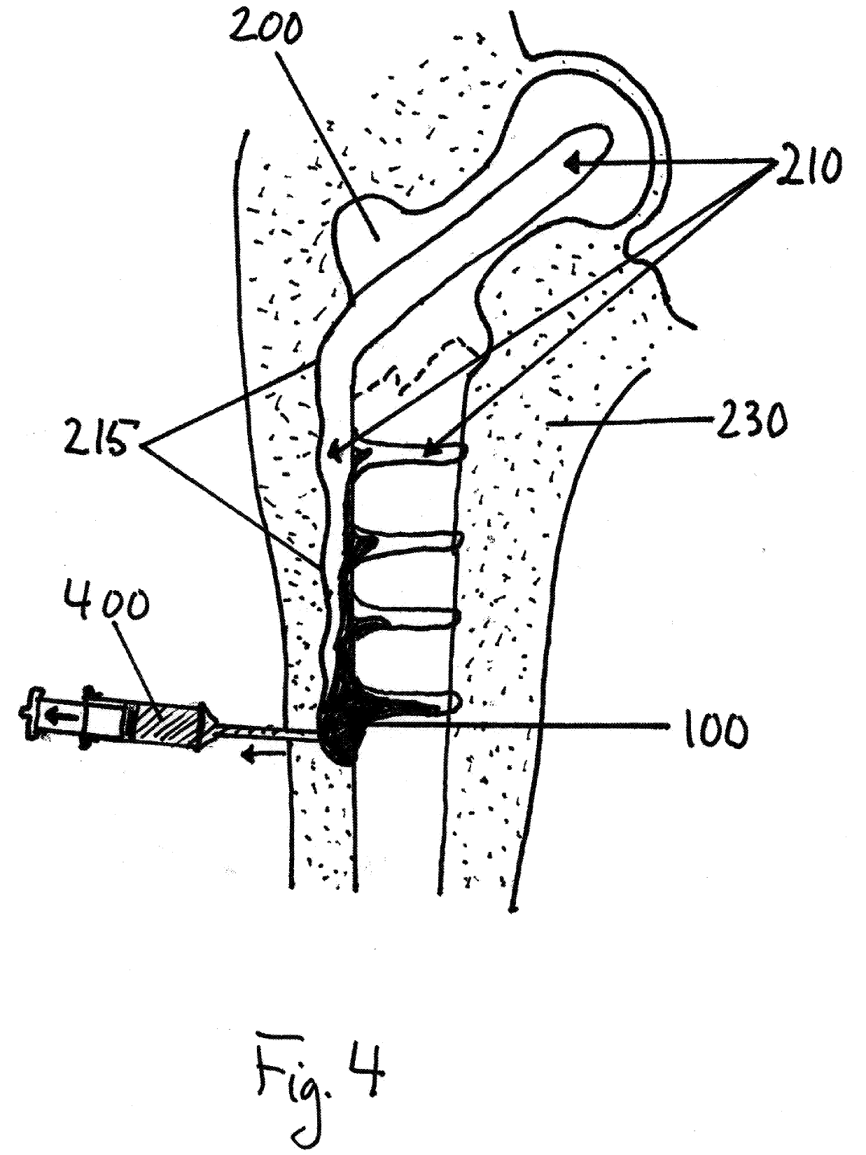

Referring to FIG. 4, as the support structure 100 is substantially removed from cavity 210 in bone 200 and the space defined by pseudo-membrane 215 in soft tissue 230, only insignificant residual amounts of the material constituting support structure 100 are left in cavity 210, the cavities left where the fasteners were, and the space defined by pseudo-membrane 215. Flushing and suction may be applied to remove a maximum amount of the substantially fluid support structure 100. Pseudo-membrane 215 and cavity 210 provide a restricted cavity preventing the substantially fluid composite material from diffusing into soft tissue 230. Solvents and additional heat may be applied to aid the process of removing substantially all of the support structure 100.

Endoscopy and/or radiology may be employed to maximize the amount of composite material removed from the subject organism. To enhance endoscopic/radiological removal, the composite material, and/or any of the components the composite material comprises, may include additives used to signal a contrast between the composite material and the surrounding body tissues of the subject organism. For example, the composite material may include additives that impart a distinctive color or texture that contrasts with other body tissue in visual endoscopy, making the composite material easy to see during the removal process, or the composite material may include a radiological contrast agent, making it visible during radiological evaluations.

Referring to FIG. 5, in some embodiments support structure 100 may be a plate which lies adjacent a fracture 150 and is attached to bone 200 via fasteners 110. Fracture 150 is supported by support structure 100 and fracture fastener 115. In these embodiments, support structure 100 is not inserted into a cavity in bone 200. Such embodiments are suitable for, e.g., certain fractures of a human humerus.

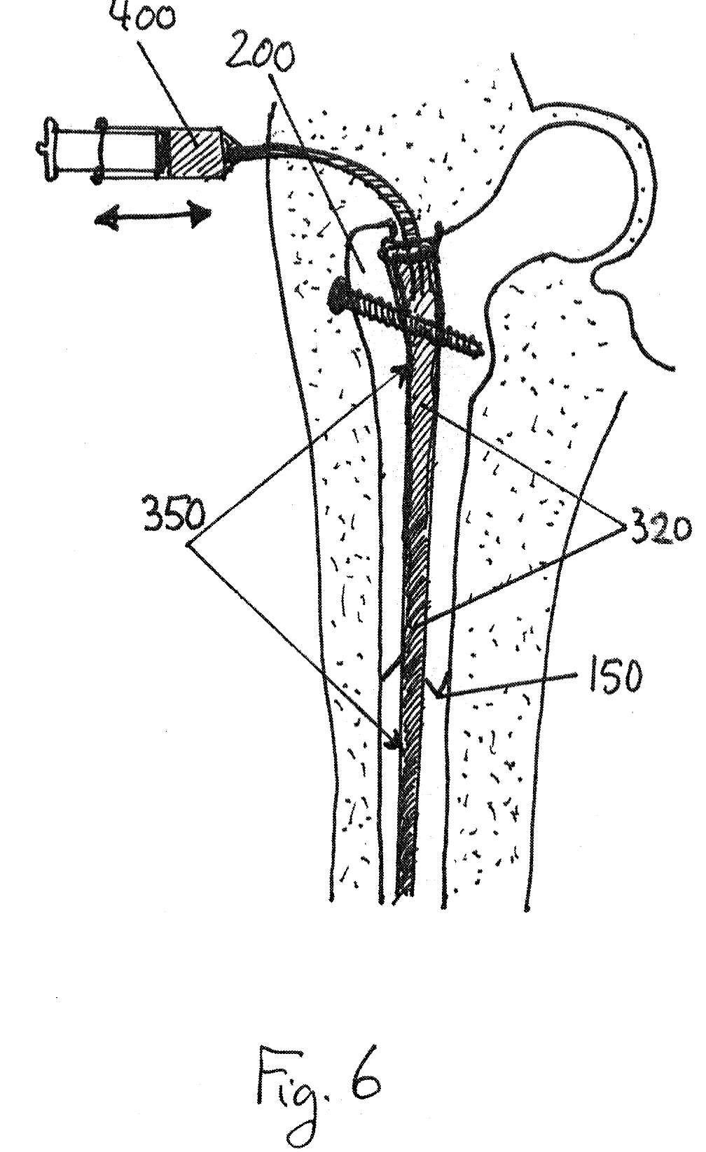

Referring to FIG. 6, in some embodiments, composite material 320 may be applied in a substantially fluid form to bone marrow or bone cavity 350 within bone 200, whereafter the composite material hardens into a substantially solid phase to provide adequate support to the fracture 150. Composite material 320 may be applied to bone marrow or bone cavity 350 via a needle, syringe, or special portal 400. As with other embodiments, composite material 320 may also be removed from the subject organism in a substantially fluid phase via a needle, syringe, or special portal 400. Composite material 320 may also be applied in a substantially fluid form to a subject organism via a device or method compatible with the disclosure of U.S. Pat. No. 5,827,289 to Reiley et al., as discussed above. More specifically, in some embodiments, composite material 320 may be applied to a bone via a balloon that is inflated: (1) within a preformed cavity of the bone or (2) to create a cavity within the bone by compressing cancellous bone and marrow against the inner cortex.

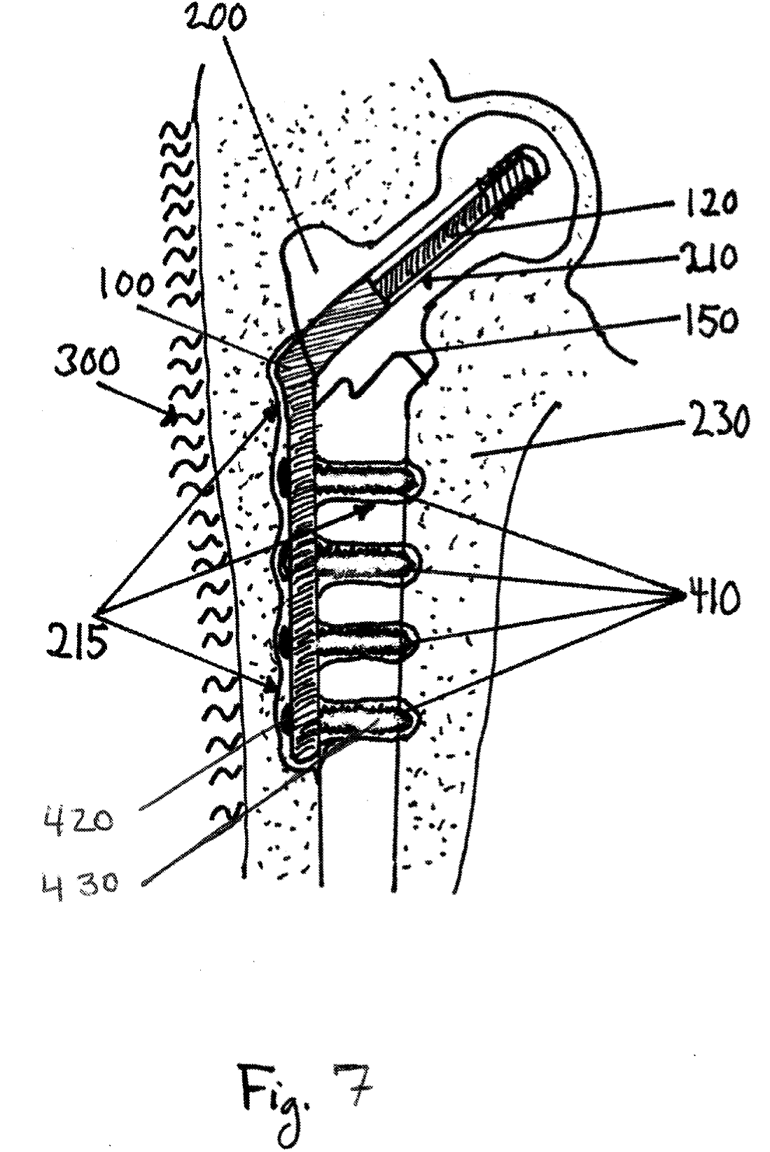

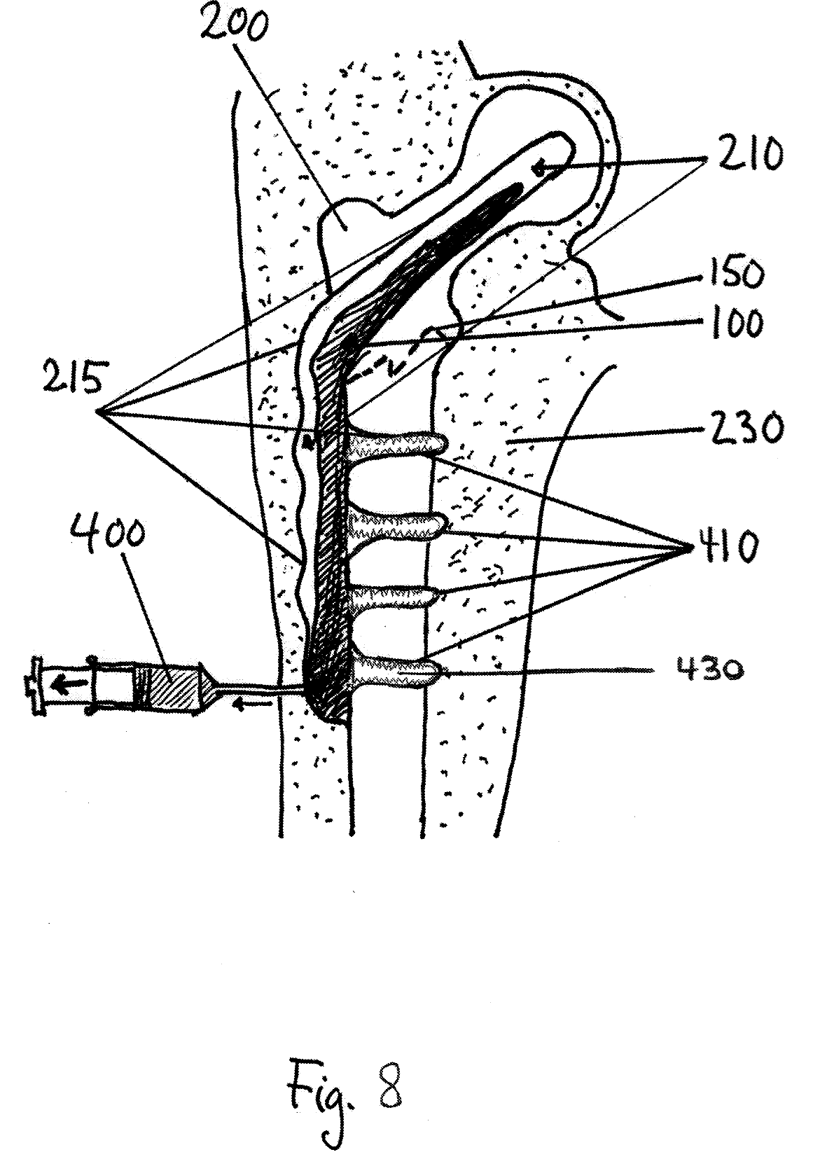

Referring to FIGS. 7 and 8, in some embodiments, support structure 100 may be attached to bone 200 with at least one fastener 410 that includes a head portion 420 and a shaft portion 430 wherein the head portion of the fastener comprises a material capable of being transformed in vivo into a substantially fluid phase by absorbing energy and the shaft portion comprises a material that remains in a substantially solid phase. In other words, the material of the head portion of the fastener is different than the material of the shaft portion. The material of the head portion may be identical to, similar to, or different than the composite material of support structure 100. In preferred embodiments, the material of the head portion is identical to or similar to the composite material of support structure 100. Employing different materials in the head and shaft portions of the fastener(s) allows the head portion to be converted from a substantially solid phase into a substantially fluid phase while the shaft portion remains in a substantially solid phase. In these embodiments, the head portion of the fastener(s) may be transformed into a substantially fluid phase at the same time support structure 100 is transformed into a substantially fluid phase and all the substantially fluid material of the fastener(s) and the support structure may be removed concurrently, leaving the shaft portion of the fastener(s) left in bone 200.

In some embodiments, the support structure can be prefabricated in such a way as to advantageously include more than one component comprising a different polymer matrix, each polymer matrix having different physical properties. For example, the support structure can be prefabricated with: a first component comprising a first biocompatible polymer matrix capable of being transformed in vivo into a substantially fluid phase by absorbing energy and melting at a first melting point; a second component comprising a second biocompatible polymer matrix capable of being transformed in vivo into a substantially fluid phase by absorbing energy and melting at a second melting point less than said first melting point, and additional components capable of strengthening said biocompatible polymer matrices and/or absorbing energy. Such a support structure may include a central, interior, or core portion made of the first component with the first polymer matrix and an outer, exterior, or peripheral portion made of the second component with the second polymer matrix having a relatively lower melting point. In applying such a support structure to a fracture, such as the ones shown in FIGS. 1 and 6, where part of the support structure is inserted into a cavity in a bone, energy may be applied to the support structure during the application procedure (as opposed to during a removal procedure) to soften or melt only the polymer matrix of the outer, exterior, or peripheral portion, while not affecting the central, interior, or core portion, thereby providing a better fit of the outer surface of the support structure with the bone cavity.

It is to be understood that the present invention is by no means limited only to the particular constructions herein disclosed and shown in the drawings. The appended claims should be construed broadly to cover any variations or modifications within the scope or range of equivalents of the claims.

We claim:

1. A composite material for surgical use in bone fracture healing comprising:
a first component comprising a biocompatible polymer matrix capable of being transformed in vivo into a substantially fluid phase by absorbing energy at a chosen time.

2. The composite material of claim 1 further comprising a second component capable of strengthening said biocompatible polymer matrix.

3. The composite material of claim 2 further comprising at least a third component capable of at least one of strengthening said biocompatible polymer matrix and increasing said energy absorbance.

4. The composite material of claim 3, wherein at least one of said second component and said third component comprise at least one of a particle, a flake, a fiber, a clay, and a nanostructure.

5. The composite material of claim 4, wherein said nanostructure is a nano-clay.

6. The composite material of claim 1, wherein said biocompatible polymer matrix comprises at least one of: (1) polycaprolactone and (2) a polycaprolactone polyol.

7. The composite material of claim 1, where said biocompatiable polymer matrix comprises polycaprolactone and a polycaprolactone polyol.

8. The composite material of claim 5, wherein said biocompatible polymer matrix comprises at least one of: (1) polycaprolactone and (2) a polycaprolactone polyol.

9. The composite material of claim 5, where said biocompatiable polymer matrix comprises polycaprolactone and a polycaprolactone polyol.

10. A support structure for bones of a living organism comprising the material of claim 7.

11. A fastener used to attach the support structure of claim 10 to a bone comprising:
a head portion and a shaft portion,
wherein said head portion comprises a material capable of being transformed in vivo into a substantially fluid phase by absorbing energy.

12. A temporary biocompatible support structure for aiding bone fracture osteosynthesis in a living organism comprising:
a polymer matrix, and
at least one component capable of strengthening said polymer matrix,
wherein said support structure is attached to a bone in a living organism,
wherein said support structure is substantially solid at a body temperature of said living organism and is substantially fluid when heated to a temperature above said body temperature in vivo, and
wherein said support structure is removable in a substantially fluid phase from said living organism.

13. The support structure of claim 12, wherein said living organism is a mammal and said body temperature is a temperature within a range of approximately 34° C. to approximately 42° C.

14. The support structure of claim 13, wherein said mammal is a human.

15. The support structure of claim 14, wherein said bone contains a cavity and said support structure comprises a portion that is insertable within said cavity.

16. The support structure of claim 15, wherein said at least one component capable of strengthening said polymer matrix is a nano-clay.

17. The support structure of claim 12, wherein said polymer matrix comprises at least one of: (1) polycaprolactone and (2) a polycaprolactone polyol.

18. The composite material of claim 12, where said polymer matrix comprises polycaprolactone and a polycaprolactone polyol.

19. The support structure of claim 16, wherein said polymer matrix comprises at least one of: (1) polycaprolactone and (2) a polycaprolactone polyol.

20. The composite material of claim 16, where said polymer matrix comprises polycaprolactone and a polycaprolactone polyol.

21. A temporary biocompatible support structure for aiding bone fracture osteosynthesis in a living organism comprising:
a polymer matrix, and
at least one component capable of strengthening said polymer matrix,
wherein said support structure is attached to a bone in a living organism,
wherein said support structure is substantially solid when applied to said living organism and said support structure can be transformed in vivo into a pulverized state by absorbing energy, and
wherein said support structure is removable from said living organism.

22. The temporary biocompatible support structure of claim 21, wherein said support structure can be transformed in vivo into a pulverized state by absorbing shock waves.

23. The temporary biocompatible support structure of claim 22, wherein said at least one component capable of strengthening said polymer matrix is a nano-clay.

24. The temporary biocompatible support structure of claim 21, wherein said polymer matrix comprises at least one of: (1) polycaprolactone and (2) a polycaprolactone polyol.

25. The temporary biocompatible support structure of claim 21, where said polymer matrix comprises polycaprolactone and a polycaprolactone polyol.

26. The temporary biocompatible support structure of claim 23, wherein said polymer matrix comprises at least one of: (1) polycaprolactone and (2) a polycaprolactone polyol.

27. The temporary biocompatible support structure of claim 23, where said polymer matrix comprises polycaprolactone and a polycaprolactone polyol.
